(19) United States
(12) Patent Application Publication
(10) Pub. No.: US 2008/0183039 A1
(43) Pub. Date: Jul. 31, 2008

(54) Balloon Positioning System for Endoscopic Access

(75) Inventors: Gary Long, Cincinnati, OH (US); David Stefanchik, Morrow, OH (US); James Spivey, Loveland, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: ETHICON ENDO-SURGERY, INC., Cincinnati, OH (US)

(21) Appl. No.: 11/627,428

(22) Filed: Jan. 26, 2007

(51) Int. Cl. A61B 1/00

(52) U.S. Cl. 600/117

(57) ABSTRACT

Devices and methods for endoscopic and/or laparoscopic surgery are provided. In one aspect, a surgical device is provided that includes an elongate member, such as an endoscope, having proximal and distal ends and a working channel associated therewith. An expandable member is also associated with the endoscope, and the expandable member is configured to be selectively expanded to engage at least a portion of a cavity within a patient, thereby stabilizing the endoscope within the patient.

### FIELD OF THE INVENTION

The present invention relates to surgical devices, and in particular to devices and methods for laparoscopic and/or endoscopic surgery.

### BACKGROUND OF THE INVENTION

Laparoscopic surgery is one type of minimally invasive surgery in which a surgeon uses numerous trocar ports to access a tissue site of interest within the abdominal cavity of a patient. Endoscopic surgery is another type of minimally invasive surgery that affords another way to access the abdominal cavity via natural openings (e.g., mouth, anus, vagina, and urethra) of the body.

General surgeons, gastroenterologists, and other medical specialists routinely use flexible endoscopes for intralumenal examination and treatment of the upper gastrointestinal (GI) tract via the mouth. In these procedures, the physician advances the flexible endoscope through the tortuous passageway of the upper GI past the pharynx, through the esophagus and gastro esophageal junction, and into the stomach. Once within the stomach, transgastric surgical procedures can be effected by positioning the endoscope at a desired location to form an access port through the stomach wall. The device can then be advanced through this access port to allow a surgeon access to other parts of the abdominal cavity. Because it can be difficult to stabilize the endoscope within the stomach to form the access port and advance it through the access port, there is a need for improved devices and methods for laparoscopic and/or endoscopic surgery.

### SUMMARY OF THE INVENTION

The present invention provides devices and methods for laparoscopic and/or endoscopic surgery. In one aspect, a surgical device includes an elongate member, such as an endoscope, having proximal and distal ends and a working channel associated therewith. An expandable member is also associated with the endoscope, and the expandable member is configured to be selectively expanded such that it engages at least a portion of a cavity within a patient to effect stabilization of the endoscope. In one embodiment, the expandable member can be formed on a sheath that is adapted to surround at least a portion of the endoscope or, in another embodiment, the expandable member can be formed on the endoscope itself.

A variety of techniques can be used to effect expansion of the expandable member, however generally a passageway is connected to the expandable member for delivering an expansion fluid thereto. By way of non-limiting example, where the expandable member is formed on a distal end of the endoscope, the endoscope can include at least one passageway. The passageway can extend from a proximal end of the endoscope to an inlet of the expandable member, and is effective for delivering an expansion fluid to the expandable member. In another embodiment, where the expandable member is formed on a sheath that surrounds the endoscope, the sheath can include at least one passageway that extends from a proximal end of the sheath to an inlet of the expandable member. In an embodiment where the device has three expandable members, each of the expandable members can have a dedicated passageway such that each expandable member is configured to expand independently.

One skilled in the art will appreciate that the expandable member(s) can have a variety of configurations, and in one embodiment it can be a balloon. The expandable member(s) can also be adapted to expand to a pre-shaped configuration and/or it can be formed from a radioopaque material.

Methods for deploying an elongate member, such as an endoscope, are also provided. In one aspect, a method for deploying an endoscope includes delivering an endoscope having an expandable member associated therewith to a target position within a patient, and selectively expanding the expandable member to engage at least a portion of a cavity within the patient. This is effective to stabilize the endoscope at the target position within the patient. As noted above, a variety of techniques can be used to facilitate expansion of the expandable member. In one embodiment, the expandable member can be expanded by delivering a fluid through a passageway that extends from a proximal end of the endoscope to an inlet of the expandable member. In another embodiment, where the expandable member is formed on a sheath that is positioned on at least a portion of the endoscope, the expandable member can be expanded by delivering a fluid through a passageway that extends from a proximal end of the sheath to an inlet of the expandable member. Once the endoscope is stabilized in position, the method can also include delivering a cutting tool through a working channel in the endoscope. In other embodiments, the method can include visualizing a position of the expandable member within the patient.

In another embodiment, where first and second expandable members are formed on the endoscope, the method can include selectively expanding the first expandable member independently of the second expandable member. The method can also include directing a distal portion of the endoscope to the target position by expanding the first expandable member to a first volume, and expanding the second expandable member to a second volume, the second volume being different than the first volume.

### BRIEF DESCRIPTION OF THE DRAWINGS

The invention will be more fully understood from the following detailed description taken in conjunction with the accompanying drawings, in which:

FIG. 1A is a perspective view of one embodiment of a surgical device positioned within a stomach of a patient with an expandable member in a deflated position;

FIG. 1B is a perspective view of the device of FIG. 1A with the expandable member in an expanded position;

FIG. 1C is a perspective view of an outer sheath of the device of FIGS. 1A and 1B;

FIG. 2 is a perspective view of another embodiment of a surgical device positioned within a stomach of a patient with an expandable member in an expanded position;

FIG. 3 is a perspective view of yet another embodiment of a surgical device positioned within a peritoneal space of a patient with an expandable member in an expanded position;

FIG. 4A is a perspective view of yet another embodiment of a surgical device positioned within a stomach of a patient with an expandable member in a deflated position; and

FIG. 4B is a perspective view of the device of FIG. 4A with the expandable member in an expanded position.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides devices and methods for deploying an elongate member, such as an endoscope. In one embodiment, the device includes an elongate member having at least one expandable member associated therewith. For example, the expandable member can be formed on a sheath that surrounds the elongate member or it can be formed on a distal portion of the elongate member. In use, the device can be delivered to a target position within a patient, and the expandable member(s) can be expanded to engage at least a portion of a cavity or hollow organ within the patient. In one embodiment and when expanded, the expandable member is in continuous direct contact with the cavity. As a result, the elongate member is stabilized within the cavity such that buckling and/or lateral movement of the elongate member within the cavity is substantially prevented. For example, should the expandable member be filled with a fluid such as water, the weight imparted to that section of the elongate member will allow more force to be applied at the tip of the elongate member without dislodging it. Additionally, expansion of the expandable member(s) can be useful to navigate the elongate member, for example, to direct it towards a desired location within the patient, as will be discussed in more detail below. Once the elongate member is stabilized at the desired location within the patient, a surgical tool, such as a cutting tool, can be inserted through a working channel associated with the elongate member for use during a surgical procedure. While the exemplary embodiment illustrates the device being used in the context of trans-gastric surgeries, one skilled in the art will appreciate that it can be used in a variety of endoscopic and laparoscopic procedures. One skilled in the art will also appreciate that the present invention has applications in robotic-assisted surgery.

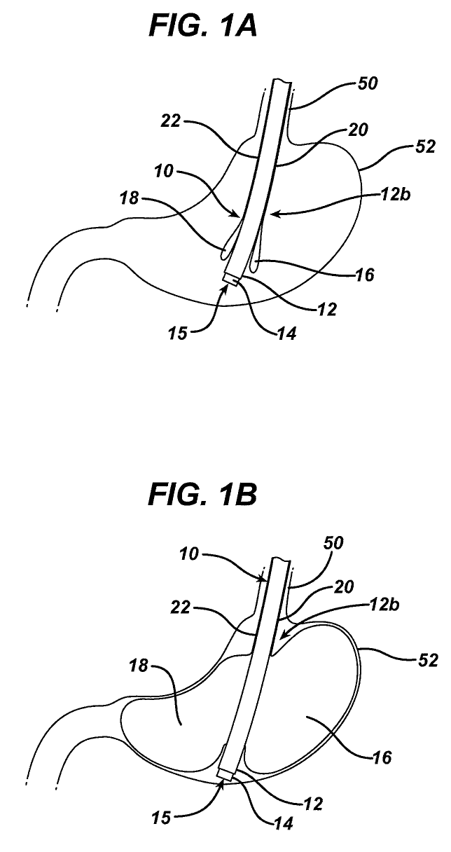

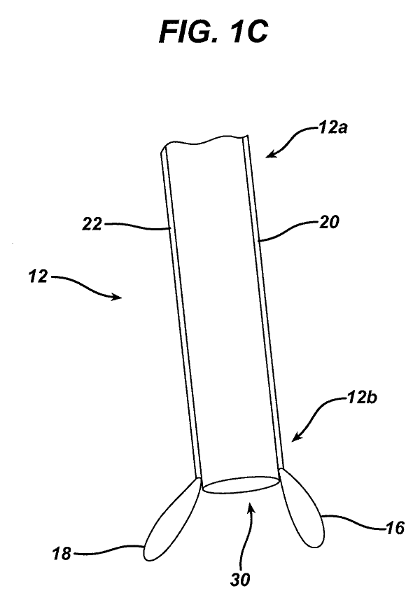

FIGS. 1A-1B illustrate one embodiment of a surgical device 10 that includes an elongate member 14 having proximal and distal ends (not labeled). A sheath 12 surrounds at least a portion of the elongate member 14, and at least one expandable member (expandable members 16, 18 are shown) is formed on the distal portion 12b of the sheath 12. FIG. 1A illustrates the device 10 in the delivery configuration, in which the expandable members 16, 18 are in the deflated position. Expandable members 16, 18 are illustrated in an exaggerated size in FIG. 1A. One skilled in the art will appreciate that the expandable members 16, 18 can be constricted such that they are essentially indistinguishable from the outer surface of the elongate member 14 when they are in the deflated position. Following the placement of the device 10 within the patient, the expandable members 16, 18 can be selectively inflated to engage a cavity within a patient, and anchor or stabilize the elongate member 14 within the patient. As shown in FIG. 1B, the expandable members 16, 18 can expand to conform to the shape of the target area, as will be discussed in more detail below. When expanded the expandable members 16, 18 are adapted to surround an outer periphery of any insertion members (e.g., the elongate member 14 and the sheath 12).

The elongate member 14 can have virtually any configuration that allows it to be laparoscopically or endoscopically inserted to a surgical site. The member 14 can be substantially cylindrical and sufficiently strong, yet flexible enough to bend during insertion through a tortuous lumen. In one embodiment, the elongate member 14 is preferably substantially solid to provide structural support, but can include a working channel 15 associated therewith. Additionally, the elongate member 14 can include markings (not shown) to facilitate visualization and placement of the device 10 within the patient. While the member 14 can have a variety of sizes, the size of the member 14 generally depends upon the desired application of the device 10. In an exemplary embodiment, where the device 10 is used during endoscopic trans-gastric surgeries, the member 14 can have a diameter in the range of about 5.0 mm to 15.0 mm. One skilled in the art will appreciate that a variety of elongate members can be used with the device of the present invention, such as an endoscope or a catheter, as are known in the art.

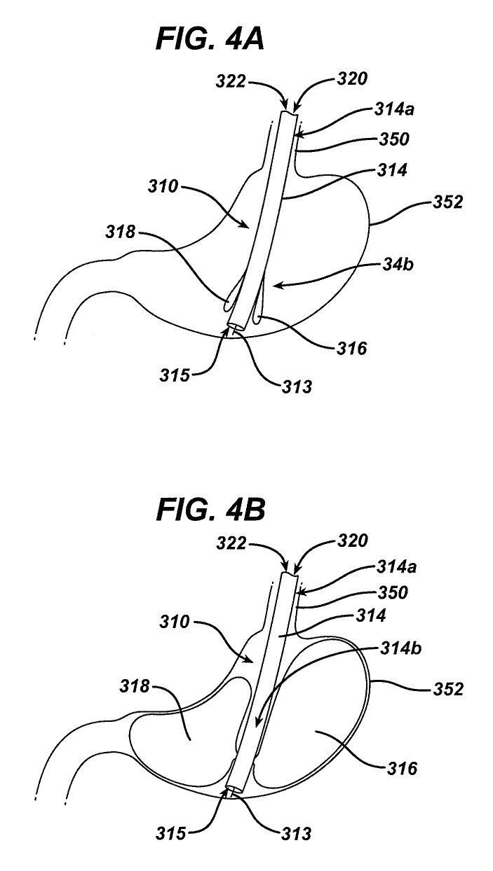

As noted above, a working channel 15 can be associated with the elongate member 14. Generally the working channel can be of a type that is commonly used in an endoscope, and can be formed in the elongate member and extend therethrough. However, in one embodiment, at least one of the working channels can be an accessory channel, as is known in the art. The working channel 15 can have a variety of shapes and sizes, but in an exemplary embodiment it has a diameter that is smaller than a diameter of the elongate member 14 so as not to interfere with the structural integrity thereof, but large enough such that surgical tools can be delivered therethrough to the target site. In another embodiment, the elongate member 14 can also have at least one other passageway that is formed therein and adapted to deliver an expansion fluid to the expandable member, as will be discussed in more detail below with respect to FIGS. 4A-4B.

As noted above, at least one expandable member is associated with the elongate member. The expandable member(s) can be formed on a sheath that surrounds the elongate member or it can be appended to a distal portion of the elongate member. FIG. 1C illustrates one embodiment of a sheath 12 that is adapted to be positioned around the elongate member 14 having expandable members 16, 18 formed on a distal portion 12b thereof. Similar to the elongate member 14, the sheath 12 can have any configuration that allows it to be laparoscopically or endoscopically inserted to a surgical site. However, the sheath 12 generally has a configuration that corresponds to the shape of the elongate member 14. For example, when the elongate member 14 is substantially cylindrical, the sheath 12 can also be substantially cylindrical. The sheath 12 can also be substantially solid with an opening 30 formed therein and extending therethrough that is adapted to receive the elongate member 14. The opening 30 should correspond in size to the diameter of the elongate member 14, such that when the sheath 12 is positioned around the elongate member 14 there is minimal space between the outer surface of the elongate member 14 and the opening 30. This minimizes the lateral movement of the elongate member 14 relative to the sheath 12, however still allows the elongate member 14 to be advanced longitudinally through the sheath 12 and into tissue, as will be discussed in more detail below.

The sheath can also have a variety of other features. In one embodiment, the sheath can have at least one passageway formed thereon or extending therethrough for delivering an expansion fluid to the expandable member, as will be discussed in more detail below. Additionally or alternatively, the sheath 12 can also have markings (not shown) formed thereon to facilitate visualization of the device 10. While the sheath 12 can have a variety of sizes, it should have a length that is effective to surround at least a portion of the elongate member 14.

One skilled in the art will appreciate that the sheath can be formed from a variety of biocompatible materials, however in one embodiment at least a portion of the sheath is formed from an elastic material. This is effective to form an expandable bladder or member, as will be discussed in more detail below.

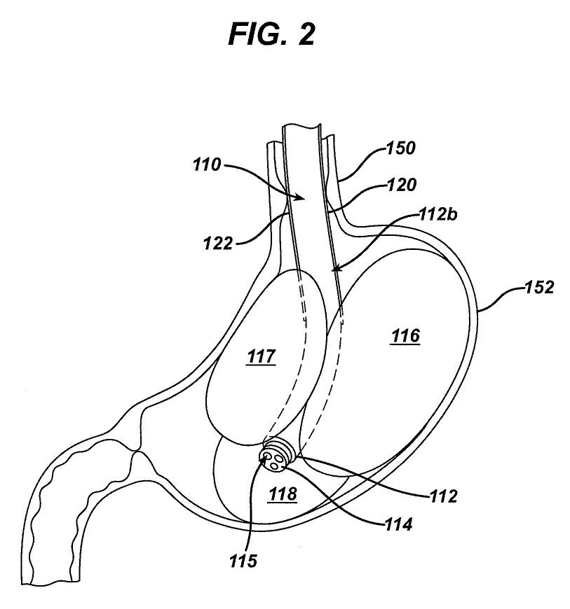

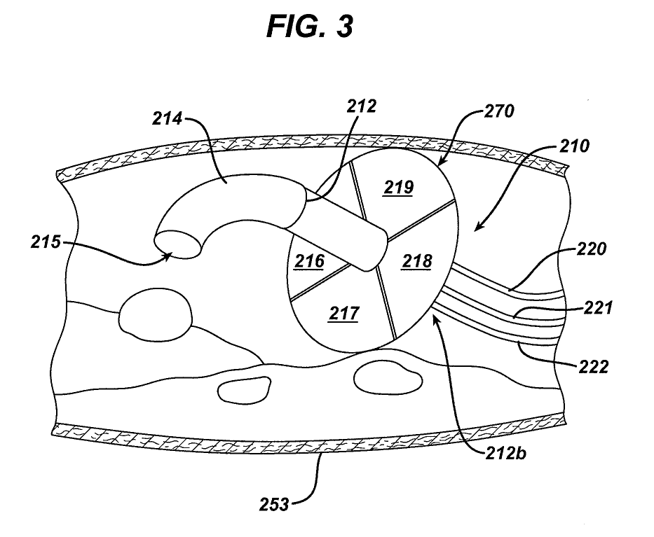

The distal portion of the sheath includes at least one expandable member, and the sheath can have any number of expandable members arranged in a variety of configurations. As noted above, FIGS. 1A-1C illustrate a sheath 12 having two expandable members 16, 18 that are formed on the distal portion 12b thereof. As shown, the expandable members 16, 18 are arranged equidistantly around the circumference of sheath 12. However in other embodiments, the expandable members can be positioned laterally or longitudinally adjacent to one another on the sheath. FIGS. 2 and 3 illustrate devices 110, 210 that have three and four expandable members 116, 117, 216, 217, 218, 219, respectively, arranged equidistantly around the circumference of the sheath 112, 212, as will be discussed in more detail below.

One skilled in the art will appreciate that a variety of techniques can be used to couple the expandable member to the sheath. In one embodiment, adhesives such as glue are used. Alternatively, a sleeve can be formed around the distal portion of the sheath to couple the expandable members thereto, or a fastener(s) can be used to hold the expandable member on the sheath. In other embodiments, the expandable members can be bladders that are formed as part of the sheath.

The expandable member(s) can have a variety of configurations, however is generally adapted to selectively expand to a preshaped configuration upon the receipt of fluid. By way of non-limiting example, when the device is adapted for use in trans-gastric surgery, the expandable member(s) can be shaped to engage the stomach of a patient. As shown in FIGS. 1A-1C, the expandable members 16, 18 have an oblong shape that corresponds to the shape of the cavity or hollow organ that they are designed to engage, however they can have other shapes. For example, FIG. 3 illustrates a device where the expandable members 216, 217, 218, 219 have an annular shape, as will be discussed in more detail below. Moreover, while the expandable members 16, 18 of the device 10 of FIGS. 1A-1C have substantially similar configurations, in other embodiments, the expandable members can be configured differently from one another to account for the configuration of the cavity, etc. One skilled in the art will appreciate that the expandable member(s) can be formed from any material that is biocompatible and able to be expanded, such as silicone or polyurethane. The expandable member(s) can also be formed from a radioopaque material to facilitate visualization thereof. In one embodiment, the expandable member(s) can be a balloon.

As noted above, a fluid can be delivered to the expandable member(s) to effect expansion thereof. A variety of techniques can be used to deliver fluid to the expandable member(s), however in one embodiment, and still referring to FIG. 1C, a passageway 20, 22 can extend from the proximal end 12a of the sheath 12 to an inlet (not shown) of the expandable member 16, 18. The device 10 can have any number of passageways, and the passageways can be adapted to facilitate expansion of one or multiple expandable members. In an exemplary embodiment, however each expandable member 16, 18 can have its own dedicated passageway 20, 22 such that the expandable members 16, 18 can be selectively and independently expanded.

The passageways 20, 22 can have any configuration that is effective to deliver the expansion fluid to the expandable member 16, 18, and can be formed integrally with the sheath 12 or coupled thereto. The passageways can also be formed on the inner or outer wall of the sheath. As shown in FIG. 1C, the passageways 20, 22 are substantially circular tubes that are appended to the outer wall of the sheath 12. The proximal ends of the passageways 20, 22 can be adapted to be coupled to a fluid delivery system as is known in the art. The distal ends of the passageways 20, 22 can be in fluid communication with an inlet of each of the expandable members 16, 18 to facilitate the delivery of fluid thereto. While the passageways 20, 22 can have any size, they are generally significantly smaller in diameter than the sheath, and in an exemplary embodiment have a diameter in the range of about 0.2 mm to 2.0 mm.

A variety of techniques can be used to couple the passageways 20, 22 to the sheath 12, however in an exemplary embodiment an adhesive, such as glue, is used. Alternatively, the passageways can be coupled to the sheath using a fastener(s) or a mechanical interlock. In use, a fluid can be delivered through the passageways 20, 22 and into an inlet of the expandable members 16, 18 such that the expandable members 16, 18 can be selectively and independently expanded. Independent expansion is particularly advantageous in that it enables a user to expand one of the expandable members 16 to a larger amount than the other expandable member 18 in order to manipulate the position of the elongate member 14, as will be discussed in more detail below.

One skilled in the art will appreciate that the device can include a variety of other features to facilitate expansion of the expandable member(s). In one embodiment, the device can include features to maintain the fluid within the expandable member(s). While a variety of techniques can be used, in one embodiment, a valve, such as a one-way valve, can be positioned downstream from the fluid delivery system, e.g., at or near the proximal end of the passageways or at the entrance of each expandable member. Additionally or alternatively, the device can include features that are adapted to monitor pressure within the balloon, such that the balloon is not over inflated. Exemplary pressure monitoring devices can include a transducer, as is known in the art. The transducer can positioned at any location that is downstream from the fluid delivery device, however in one embodiment it is positioned in or on the balloon. By way of non-limiting example, the transducer can be adapted to surround the inlet of the expandable member(s), such that it can measure the pressure of the fluid within the expandable member(s). Alternatively, the transducer can be positioned at an inlet of a proximal end of the expandable member to monitor the inlet pressure. In other embodiments, a strain gage can be mounted on the surface of the expandable member to monitor the pressure of the expandable member.

FIG. 2 illustrates another embodiment of a device 110 for deploying an endoscope. The device 110 of FIG. 2 is similar to the device 10 of FIGS. 1A-1C, and has an elongate member 114 that is surrounded by a sheath 112. However, three expandable members 116, 117, 118 are arranged on the distal portion 112b of the sheath 112. The three expandable members 116, 117, 118 can be arranged in a variety of configurations, however as shown they are positioned equidistantly from one another around the circumference of the sheath 112. The expandable members 116, 117, 118 can also have any shape and size, however as shown they are all oblong, and approximately the same size. The device 110 also includes three passageways (passageways 120 and 122 are shown) that extend from a proximal end 112a of the sheath 112 to inlets (not shown) formed in each of the expandable members 116, 117, 118. In use, an expansion fluid can be delivered to the passageways 120, 122 and through an inlet of each of the expandable members 116, 117, 118 to facilitate placement and stabilization of the elongate member 114.

FIG. 3 illustrates yet another embodiment of a device 210 for deploying an endoscope such that the weight provided by the expandable members 216, 217, 218, 219 anchors the elongate member 14 so that a tip thereof can be articulated within the peritoneal space 253. The device 210 of FIG. 3 is similar to the device 10 of FIGS. 1A-1C, and has an elongate member 214 that is surrounded by a sheath 212. However, the device 210 includes four expandable members 216, 217, 218, 219 that are formed on the distal portion 212b of the sheath 212. The expandable members 216, 217, 218, 219 can be arranged in a variety of configurations, however as shown they are positioned equidistantly from one another around the circumference of the sheath 212. The expandable members 216, 217, 218, 219 can also have any shape and size, however as shown they are all approximately the same shape and size. The device 210 also includes four passageways (passageways 220, 221, 222 are shown) that extend from a proximal end of the sheath 212 to inlets (not shown) formed in each of the expandable members 216, 217, 218, 219. In use, and as noted above with respect to FIGS. 1A-2, the expandable members 216, 217, 218, 219 can be expanded selectively and independently of one another. As a result, and as shown, all of the expandable members 216, 217, 218, 219 can be expanded to the same amount to effectively form a ring 270 around the distal portion 212b of the sheath 212. However, in other embodiments, one skilled in the art will appreciate that the expandable members can be expanded to varying degrees.

As noted above, a variety of other techniques can be used to associate the expandable member(s) with the elongate member, and in other embodiments, the expandable member(s) can be coupled to a distal portion of the elongate member. FIGS. 4A-4B illustrate one embodiment of a device 310 having an elongate member 314 with expandable members (expandable members 316, 318 are shown) that are coupled to a distal portion 314b thereof. FIG. 4A illustrates the device 310 in the delivery configuration, in which the expandable members 316, 318 are in the deflated position. Expandable members 316, 318 are illustrated in an exaggerated size in FIG. 4A. One skilled in the art will appreciate that the expandable members 316, 318 can be constricted such that they are essentially indistinguishable from the outer surface of the elongate member 314 when they are in the deflated position. Following the delivery and placement of the device 310 within the patient, the expandable members 316, 318 can be selectively inflated to engage a cavity within a patient, and anchor or stabilize the elongate member 314 within the patient, as shown in FIG. 4B. Additionally or alternatively, in embodiments where the device 310 is used for trans-gastric surgeries, the expandable members 316, 318 can be inflated once the elongate member 314 is positioned outside the patient's stomach to anchor the elongate member 314. As shown, when the expandable members 316, 318 are in the expanded position they surround the outer periphery of the elongate member 314.

The elongate member 314 and the expandable members 316, 318 are similar to the elongate member 14 and the expandable members 16, 18 of the device 10 of FIGS. 1A-1C. That is, the expandable members 316, 318 have a substantially oblong shape and substantially the same size. However, the expandable members 316, 318 are not coupled to a sheath, but rather are coupled to a distal portion 314b of the elongate member 314. One skilled in the art will appreciate that a variety of techniques can be used to couple the expandable members 316, 318 to the elongate member 314, such as an adhesive (e.g., glue), a fastener, or a mechanical interlock connection. While the exemplary embodiment illustrates a device having two expandable members 316, 318 formed on the distal portion 314b thereof, one skilled in the art will appreciate that the device can have any number of expandable members in any arrangement. For example, in other embodiments the device can have three or four expandable members that are arranged equidistantly around the circumference of the elongate member.

A passageway can be used to deliver an expansion fluid to the expandable member to effect expansion thereof. The device can have any number of passageways, however, in an exemplary embodiment the device can have one passageway dedicated to each expandable member such that the expandable members can be independently expanded. Thus, as shown, the device 310 can have two passageways 320, 322. The passageways can be formed integrally with the elongate member or coupled thereto. However, as shown in FIGS. 4A-4B the passageways 320, 322 are formed integrally with the elongate member 314 and extend from a proximal end of the elongate member 314a to an inlet of each of the expandable members 316, 318. In other embodiments, the passageways can be tubes that are appended to an inner or outer surface of the elongate member, similar to the passageways formed on the sheath discussed above.

As previously indicated, the devices disclosed herein can be used to perform a variety of medical procedures. In an exemplary embodiment, however, the devices can be used to facilitate insertion of an endoscope through the stomach wall during trans-gastric procedures. Following preparation of the patient as known in the art, the device 10, 110, 210, 310 can be inserted into a natural or created orifice to a target site. As noted above, the device 10, 110, 210, 310 is typically inserted in the delivery configuration shown in FIGS. 1A and 4A, where expandable members 16, 18, 116, 117, 118, 216, 217, 218, 219, 316, 318 are in the deflated position. For example, as shown in FIGS. 4A-4B, where the device 310 includes expandable members 316, 318 that are formed on the distal end 314b of the elongate member 314, the elongate member 314 can be inserted down the esophagus 350 and into the stomach 352. A guidewire 313 can optionally be used to facilitate insertion and positioning of the device. In embodiments where the expandable members 16, 18, 116, 117, 118, 216, 217, 218, 219 are formed on a sheath 12, 112, 212, such as those shown in FIGS. 1A-3, the elongate member 14, 114, 214 can be inserted down the esophagus 50, 150 and into the stomach 52, 152 (FIGS. 1A-2) and/or through the stomach and into the peritoneal space 253 (FIG. 3), as discussed below. The sheath 12, 112, 212 can subsequently be slid over the elongate member 14, 114, 214. Alternatively, the sheath 12, 112, 212 and the elongate member 14, 114, 214 can be delivered together to the stomach 52, 152 and/or peritoneal space 253, or the sheath 12, 112, 212 can be advanced into the stomach 52, 152 and/or peritoneal space 253 under fluoroscopic guidance, prior to advancing the elongate member 14, 114, 214. Similar to the technique used with respect to the device of FIGS. 4A-4B, a guidewire can also optionally be used to facilitate delivery of the devices 10, 110, 210. Additionally or alternatively, markers (not shown) located on the distal end of the elongate member and/or the sheath can be used to determine the position of the device within the patient and relative to the tissue. In other embodiments, where the expandable member is formed from a radioopaque material, a user can visualize the expandable member to determine the location of the device relative to the tissue using fluoroscopic methods as are known in the art.

Once the device 10, 110, 210, 310 is positioned at a desired location within the patient, a fluid can be delivered through the passageways 20, 22, 120, 122, 220, 221, 222, 320, 322 to an inlet of the each of the expandable members 16, 18, 116, 117, 118, 216, 217, 218, 219, 316, 318. A variety of biocompatible fluids can be used to inflate the expandable member, such as water, saline, or air. In other embodiments, the fluid can be radioopaque to facilitate visualization of the device, as noted above. The expandable members can be expanded simultaneously or at different times, depending upon the needs of the user. During and after expansion, the volume of the expandable members can also be monitored using a transducer associated with the expandable members, as noted above.

For example, and referring to the device 10 shown in FIGS. 1A-1C, a fluid can be delivered through the passageways 20, 22 formed on the sheath 12 to the expandable members 16, 18, such that the expandable members 16, 18 are expanded to a volume that is sufficient to engage the cavity (e.g., the stomach 52 or, in other embodiments, the peritoneal cavity, as discussed below). Thus, depending upon the configuration of the expandable members and the shape and size of the cavity, the expandable members can be expanded to the same volumes or different volumes to stabilize the elongate member within the cavity. By way of non-limiting example, the fluid can be delivered the first expandable member 16 such that it is expanded to a first volume. Fluid can be simultaneously delivered to the second expandable member 18 and the expandable member 18 can be expanded to a second volume that is different from the volume of the first expandable member 16. Alternatively, the fluid can be delivered to the second expandable member 18 at a different time, and the expandable member 18 can be expanded to the second volume. When expanded, the expandable members 16, 18 surround the outer periphery of the elongate member 14, and the weight of fluid anchors the expandable members 16, 18 within the cavity 52, such that a tip of the elongate member 14 can be manipulated, for example.

The expansion of the expandable members 16, 18 is also effective to facilitate placement of the elongate member. When the expandable members 16, 18 are expanded to volumes that are different from one another, as noted above, the difference in size between the expandable members 16, 18 can cause the elongate member 14 to be pushed a distance away from the first expandable member 16. This allows a user to place the device at a desired location within the stomach 52, as well as anchor or move the elongate member within or throughout the rest of the abdominal cavity.

In embodiments where there are three or four expandable members, the expandable members can also be independently expanded to the same or different amounts depending upon the configuration of the expandable members and the shape and size of the cavity. As shown in FIG. 2, where the device 110 has three expandable members 116, 117, 118 that have substantially similar configurations each of the expandable members 116, 117, 118 are expanded to substantially the same amount to engage the cavity. As a result, the distal portion 114b of the elongate member 114 is stabilized within the stomach 152. When the device 210 has four expandable members 216, 217, 218, 219 that have substantially similar configurations, as shown in FIG. 3, each of the expandable members 216, 217, 218, 219 are expanded to a volume that is effective to form a substantially continuous ring 270 around the distal portion 214b of the elongate member 214. This ring 270 is effective to provide an anchor for the elongate member 214 within the peritoneal space 253, as will be discussed in more detail below. In other embodiments, however the expandable members of the devices of FIGS. 2-3 can be expanded to different volumes to facilitate placement of the elongate member. One skilled in the art will appreciate that the expanded configuration of the device can vary depending upon the procedure performed and the needs of the surgeon.

The device 310 shown in FIGS. 4A-4B can be used in a manner similar to that disclosed above with respect to the device of FIGS. 1A-1C, however fluid can be delivered through the passageways 320, 322 formed within the elongate member 314 to expand the expandable members 316, 318. The expandable members 316, 318 can be expanded prior or subsequently to movement of the elongate member 314 through the puncture in the stomach wall, as will be discussed in more detail below.

Once the elongate member 14, 114, 214, 314 is stabilized within the stomach 52, 152, 352, a cutting tool (not shown), such as a needle knife, can be inserted through the working channel 15, 115, 215, 315 in the elongate member 14, 114, 214, 314. The cutting tool can then be used to puncture the stomach wall. Following formation of the puncture, the cutting tool can be removed from the working channel 15, 115, 215, 315. The elongate member 14, 114, 214, 314 can then be inserted through the puncture in the stomach wall to access other organs within the abdominal cavity. For example, when the device 10, 110, 210 includes a sheath 12 surrounding the elongate member (as shown in FIGS. 1A-3), the elongate member 14, 114, 214 can be moved relative to the sheath 12, 112, 212 and through the opening while the expandable members 16, 18, 116, 117, 118, 216, 217, 218, 219 remain expanded. In one embodiment, the expandable members 16, 18, 116, 117, 118, 216, 217, 218, 219 can be deflated and the sheath 12, 112, 212 can be moved through the opening and into the peritoneal space. The expandable members 16, 18, 116, 117, 118, 216, 217, 218, 219 can then be expanded again to anchor the device 10, 110, 210 in a desired position within the peritoneal space. By way of non-limiting example, FIG. 3 illustrates the device 210 anchored within the peritoneal space 253, such that the distal tip of the elongate member 214 can then be manipulated within the cavity 253.

In embodiments where the expandable members 316, 318 are formed on the distal end 314b of the elongate member 314 (FIGS. 4A-4B), the expandable members 316, 318 can be deflated as the elongate member is moved through the puncture in the stomach wall. This is particularly advantageous as the expandable members prevent the elongate member from buckling as it is inserted through the puncture. Additionally, or alternatively, as noted above, the expandable members 316, 318 can be expanded subsequently to insertion of the elongate member 314 through the puncture in the stomach wall to stabilize the elongate member 314 at a position outside of the stomach 352.

The devices 10, 110, 210, 310 disclosed herein can be designed to be disposed of after a single use, or they can be designed to be used multiple times. In either case, however, the devices 10, 110, 210, 310 can be reconditioned for reuse after at least one use. Reconditioning can include any combination of the steps of disassembly of the device 10, 110, 210, 310, followed by cleaning or replacement of particular pieces, and subsequent reassembly. In particular, the device 10, 110, 210, 310 can be disassembled, and any number of the particular pieces or parts of the device 10, 110, 210, 310 can be selectively replaced or removed in any combination. Upon cleaning and/or replacement of particular parts, the device 10, 110, 210, 310 can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

One skilled in the art will further appreciate that the system and the device described herein can be processed before surgery. First, a new or used device is obtained and if necessary cleaned. The device can then be sterilized. In one sterilization technique, the device can be placed in a closed and sealed container, such as a plastic or TYVEK bag. The container and the device are then placed in a field of radiation that can penetrate the container, such as gamma radiation, x-rays, or high-energy electrons. The radiation kills bacteria on the device and in the container. The sterilized device can then be stored in the sterile container, and the sealed container keeps the device sterile until it is opened in the medical facility. In other embodiments, the device can be sterilized using any other technique that is known in the art, such as beta or gamma radiation, ethylene oxide, steam, etc.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A surgical device, comprising:
an endoscope having proximal and distal ends and at least one working channel associated with the endoscope; and
at least one expandable member that is associated with the endoscope, the at least one expandable member configured to be selectively expanded to engage at least a portion of a cavity in a patient to effect stabilization of the endoscope.

2. The device of claim 1, wherein the at least one expandable member is formed at a distal portion of the endoscope.

3. The device of claim 2, further comprising at least one passageway that extends from a proximal end of the endoscope to an inlet of the at least one expandable member, the at least one passageway being configured to deliver an expansion fluid to the at least one expandable member.

4. The device of claim 1, wherein the at least one expandable member is formed on a sheath surrounding at least a portion of an outer surface of the endoscope.

5. The device of claim 4, wherein the sheath includes at least one passageway extending from a proximal end of the sheath to an inlet of the at least one expandable member, the at least one passageway being configured to deliver an expansion fluid to the at least one expandable member.

6. The device of claim 1, wherein the at least one expandable member is a balloon.

7. The device of claim 1, wherein the at least one expandable member is adapted to expand to a pre-shaped configuration.

8. The device of claim 1, wherein the at least one expandable member is formed from a radioopaque material.

9. The device of claim 1, having at least three expandable members.

10. The device of claim 9, wherein each of the at least three expandable members has a dedicated passageway such that each expandable member is configured to expand independently.

11. A method for deploying an endoscope, comprising:
delivering an endoscope having at least one expandable member associated therewith to a target position within a patient; and
selectively expanding the at least one expandable member to engage at least a portion of a cavity within the patient such that the endoscope is stabilized at the target position within the patient.

12. The method of claim 11, wherein the at least one expandable member is expanded by delivering a fluid through at least one passageway that extends from a proximal end of the endoscope to an inlet of the at least one expandable member.

13. The method of claim 11, wherein the at least one expandable member is formed on a sheath that is positioned on at least a portion of the endoscope.

14. The method of claim 13, wherein the expandable member is expanded by delivering a fluid through at least one passageway that extends from a proximal end of the sheath to an inlet of the at least one expandable member.

15. The method of claim 11, wherein the at least one expandable member is formed from a radioopaque material, and the method further comprises visualizing a position of the at least one expandable member within the patient.

16. The method of claim 11, further comprising delivering a cutting tool through a working channel in the endoscope once the at least one expandable member is expanded to stabilize the endoscope at the target position.

17. The method of claim 11, wherein first and second expandable members are formed on the endoscope, and the method includes selectively expanding the first expandable member independently of the second expandable member.

18. The method of claim 17, further comprising directing a distal portion of the endoscope to the target position by expanding the first expandable member to a first volume, and the second expandable member to a second volume, the second volume being different than the first volume.
